(19) United States
(12) Patent Application Publication
(10) Pub. No.: US 2026/0224205 A1
(43) Pub. Date: Aug. 6, 2026

(54) ULTRASOUND IMAGING DEVICE AND OPERATION METHOD THEREOF

(71) Applicant: Samsung Medison Co., Ltd., Hongcheon-gun, Gangwon-do (KR)

(72) Inventor: Young Min Kim, Hongcheon-gun, Gangwon-do (KR)

(73) Assignee: SAMSUNG MEDISON CO., LTD., Hongcheon-gun, Gangwon-do (KR)

(21) Appl. No.: 19/155,539

(22) PCT Filed: May 17, 2023
(86) PCT No.: PCT/KR2023/006710

§ 371 Date: Aug. 11, 2025

(30) Foreign Application Priority Data
Feb. 20, 2023 (KR) 10-2023-0022442

(51) Int. Cl. A61B 8/00

(52) U.S. Cl. CPC A61B 8/54; A61B 8/461; A61B 8/469

(57) ABSTRACT

An ultrasound imaging device according to an embodiment of the present disclosure includes: a display that displays an ultrasound image generated based on an ultrasound signal generated from a transducer of a probe and receives a user input through a touch screen; and a controller that determines whether a foreign substance has come into contact with an area of the display where the ultrasound image is displayed, wherein the controller may adjust touch sensitivity on the area of the display where the ultrasound image is displayed when it is determined that a foreign substance has come into contact with the area of the display.

### TECHNICAL FIELD

The present disclosure relates to an ultrasound imaging device and an operating method thereof.

### BACKGROUND ART

An ultrasound imaging device irradiates an object with an ultrasound signal generated from a transducer of a probe and receives information about an echo signal reflected from the object to obtain an image of the internal part of the object. In particular, ultrasound imaging devices are used for medical purposes such as observing the inside of an object, detecting foreign substances, and measuring injuries. These ultrasound imaging devices have the advantages of being more stable than imaging devices that use X-rays, being able to display images in real time, and being safe because there is no radiation exposure, and thus are widely used along with other imaging devices.

Electronic devices utilizing a touch screen are becoming more widespread. Accordingly, an ultrasound imaging device that interfaces with a user by using a touch screen has been developed.

When foreign substances (ultrasound gel, disinfectant, etc.) are present on the touch screen of the ultrasound imaging device, malfunctions such as not recognizing touches or recognizing non-touched parts may occur. When examining an object, foreign substances frequently get on the touch screen, and thus users have the inconvenience of having to frequently wipe the touch screen.

Therefore, in the ultrasound imaging device using a touch screen, there is a need to provide a method and device to enable a user to operate the device more conveniently.

### DISCLOSURE OF INVENTION

### Technical Problem

The present disclosure provides a method and device for a user to conveniently operate an ultrasound imaging device using a touch screen.

### Solution to Problem

An ultrasound imaging device according to an embodiment of the present disclosure includes: a display that displays an ultrasound image generated based on an ultrasound signal generated from a transducer of a probe and receives a user input through a touch screen; and a controller that determines whether a foreign substance has come into contact with an area of the display where the ultrasound image is displayed, wherein the controller may adjust touch sensitivity on the area of the display where the ultrasound image is displayed when it is determined that a foreign substance has come into contact with the ultrasound image.

Specifically, while a user input is received in an area with which the foreign substance has come into contact, the controller may adjust the touch sensitivity on the area of the display where the ultrasound image is displayed.

Specifically, the controller may adjust the touch sensitivity of an area that is in contact with the foreign substance.

Specifically, the controller may compare the user input received from the display with a recognition criterion for a valid touch to determine whether the user input is a valid input.

Specifically, the recognition criterion for a valid input may include a magnitude of a change in electrostatic capacitance.

Specifically, the controller may lower the recognition criterion for a valid input and process, as a valid input, a user input having a lower intensity than the recognition criterion for a valid input before the lowering on the ultrasound image.

Specifically, the display may further display a setting button to adjust the ultrasound image.

Specifically, when it is determined that a foreign substance has come into contact with the setting button, the controller may freeze the ultrasound image or cause a warning alarm window to pop up on the display.

Specifically, when it is determined that a foreign substance has come into contact with the setting button, the controller may raise the recognition criterion for a valid input, and process a user input on the setting button with an intensity greater than the recognition criterion for a valid input before the raising, as an invalid input.

Specifically, the user input may include setting a region of interest on the ultrasound image or measuring an object.

An operating method of an ultrasound imaging device, according to the present disclosure, includes: displaying an ultrasound image generated based on an ultrasound signal generated from a transducer of a probe and receiving a user input through a touch screen; and determining whether a foreign substance has come into contact with an area of a display where the ultrasound image is displayed, wherein, when it is determined that a foreign substance has come into contact with the area of the display where the ultrasound image is displayed, touch sensitivity on the area of the display may be adjusted.

Specifically, while a user input is received in an area with which the foreign substance has come into contact, the touch sensitivity on the area of the display where the ultrasound image is displayed may be adjusted.

Specifically, the touch sensitivity of an area that is in contact with the foreign substance may be adjusted.

Specifically, the user input received from the display may be compared with a recognition criterion for a valid touch, to determine whether the user input is a valid input.

Specifically, the recognition criterion for a valid input may include a magnitude of a change in electrostatic capacitance.

Specifically, the recognition criterion for a valid input may be lowered and a user input having a lower intensity than the recognition criterion for a valid input before the lowering on the ultrasound image may be processed as a valid input.

Specifically, a setting button for adjusting the ultrasound image may be further displayed.

Specifically, when it is determined that a foreign substance has come into contact with the setting button, the ultrasound image may be frozen or a warning alarm window may be caused to pop up on the display.

Specifically, when it is determined that a foreign substance has come into contact with the setting button, the recognition criterion for a valid input may be raised, and a user input on the setting button with an intensity greater than the recognition criterion for a valid input before the raising may be processed as an invalid input.

Specifically, the user input may include setting a region of interest on the ultrasound image or measuring an object.

A computer-readable recording medium including a program for executing an operating method of an ultrasound imaging device, according to the present disclosure, is provided, wherein the recording medium may include the program for executing the operating method including: displaying an ultrasound image generated based on an ultrasound signal generated from a transducer of a probe and receiving a user input through a touch screen; and determining whether a foreign substance has come into contact with an area of a display where the ultrasound image is displayed, wherein, when it is determined that a foreign substance has come into contact with the area of the display where the ultrasound image is displayed, touch sensitivity on the area of the display is adjusted.

### Advantageous Effects of Invention

According to an ultrasound imaging device and an operating method thereof, of the present disclosure, failure to receive a touch input, or an incorrect input to a touch screen when the touch screen is contaminated with foreign substances or the like may be prevented.

The effects of the present disclosure are not limited to the effects described above, and effects not described herein may be clearly understood by a person skilled in the art from the present specification and the attached drawings.

### BRIEF DESCRIPTION OF DRAWINGS

FIG. 1 is a structural block diagram of an ultrasound imaging device according to an embodiment of the present disclosure.

FIG. 2 is an enlarged view of a display including an area of a display area, in which an ultrasound image generated based on an ultrasound signal generated from a transducer of an ultrasound probe is output, according to an embodiment of the present disclosure.

FIG. 3 is a diagram illustrating measurement on a display of an area in which an ultrasound image is output based on an ultrasound signal, according to an embodiment of the present disclosure.

FIG. 4 is a diagram illustrating changing, on a display, of a region of interest of an area in which an ultrasound image generated based on an ultrasound signal is output, according to an embodiment of the present disclosure.

FIG. 5 is a diagram illustrating occurrence of a warning alarm in an ultrasound imaging device according to an embodiment of the present disclosure.

FIG. 6 is a flowchart of an operating method of an ultrasound imaging device, according to a first embodiment of the present disclosure.

FIG. 7 is a flowchart of an operating method of an ultrasound imaging device, according to a second embodiment of the present disclosure.

FIG. 8 is a flowchart of an operating method of an ultrasound imaging device, according to a third embodiment of the present disclosure.

FIG. 9 is a flowchart of an operating method of an ultrasound imaging device, according to a fourth embodiment of the present disclosure.

### MODE FOR THE INVENTION

The present specification clarifies the scope of the present disclosure and explains the principles of the present disclosure and discloses examples to enable a person skilled in the art to practice the present disclosure. The disclosed embodiments may be implemented in various forms.

Throughout the present specification, when a part is said to be “connected” to another part, this includes not only a direct connection but also an indirect connection, and an indirect connection includes a connection via a wireless communications network.

Additionally, the terminology used herein is for the purpose of describing embodiments only and is not intended to limit and/or restrict the disclosed disclosure. Singular expressions include plural expressions unless the context clearly indicates otherwise. In the present specification, terms such as “include” or “have” are intended to specify the presence of a feature, number, step, operation, component, part or combination thereof described in the specification, but do not exclude in advance the possibility of the presence or addition of one or more other features, numbers, steps, operations, components, parts or combinations thereof.

Additionally, terms including ordinal numbers such as “first,” “second,” etc., used herein may be used to describe various components, but the components are not limited by the terms, and the terms are used only for the purpose of distinguishing one component from another. For example, without departing from the scope of the present disclosure, a first component could be referred to a second component, and similarly, a second component could also be referred to as a first component.

Additionally, in the present specification, expressions such as “first,” “second,” or “first −1” are exemplary terms for referring to different components, objects, images, pixels, or patches. Therefore, the expressions “first,” “second,” or “first −1,” etc. do not indicate any order or priority among the components.

Additionally, terms such as “~unit”, “~or”, “~block”, “~member”, and “~module” may indicate a unit that processes at least one function or operation. For example, the above terms may indicate at least one hardware such as an FPGA (field-programmable gate array)/ASIC (application specific integrated circuit), at least one software stored in a memory, or at least one process processed by a processor.

The symbols attached to each operation are used to identify each operation and do not indicate the order of the operations, and the operations may be performed in a different order than stated unless the context clearly indicates a specific order.

Additionally, in the present specification, an image may include a medical image obtained by a medical imaging device such as a magnetic resonance imaging (MRI) device, a computed tomography (CT) device, an ultrasound imaging device, or an X-ray imaging device, and may also provide or control an ultrasound image and a medical image of a modality other than ultrasound.

Additionally, in the present specification, an ‘object’ refers to an object of photography and may include a person, an animal, or a part thereof. For example, the object may include a part of the body (such as an organ or system) or a phantom.

Throughout the specification, the term “ultrasound image” refers to an image of an object that is processed based on an ultrasound signal transmitted to the object and reflected from the object.

Hereinafter, the term “on an ultrasound image” or “on a setting button” refers to an area on a display, in which the ultrasound image or the setting button is displayed.

Hereinafter, embodiments according to the present disclosure will be described in detail with reference to the attached drawings.

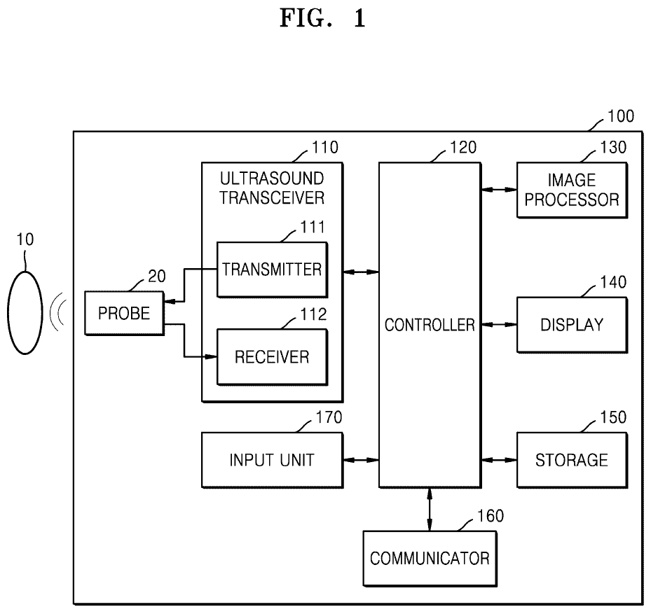

FIG. 1 is a structural block diagram of an ultrasound imaging device 100 according to an embodiment of the present disclosure.

The ultrasound imaging device 100 according to an embodiment may include a probe 20, an ultrasound transceiver 110, a controller 120, an image processor 130, a display 140, a storage 150, a communicator 160, and an input unit 170.

The ultrasound imaging device 100 may be implemented in a portable form as well as a cart form. Examples of portable ultrasound imaging devices may include, but are not limited to, smart phones, laptop computers, PDAs, tablet PCs, etc., which include probes and applications.

The probe 20 may include a plurality of transducers. The plurality of transducers may transmit ultrasound signals to an object 10 according to a transmission signal received from a transmitter 111. The plurality of transducers may receive ultrasound signals reflected from the object 10 and form a received signal. Additionally, the probe 20 may be implemented as an integral portion of the ultrasound imaging device 100, or may be implemented as a separate part connected to the ultrasound imaging device 100 via wired or wireless means. Additionally, the ultrasound imaging device 100 may include one or more probes 20 depending on the implementation form.

The controller 120 controls the transmitter 111 to form a transmission signal to be applied to each of the plurality of transducers by considering positions and focus points of the plurality of transducers included in the probe 20.

The controller 120 controls a receiver 112 to generate ultrasound data by converting an analog-to-digital reception signal received from the probe 20 and adding the digitally converted reception signals while considering the positions and focus points of the plurality of transducers.

The image processor 130 generates an ultrasound image by using ultrasound data generated by the ultrasound receiver 112.

Meanwhile, ultrasound images may represent the movement of an object as a Doppler image as well as a gray scale ultrasound image which is a result of scanning an object according to A mode (amplitude mode), B mode (brightness mode), and M mode (motion mode).

A-mode is the most basic form of ultrasound image display method and is a method of displaying the intensity of reflected sound as amplitude on the time (distance) axis. When the reflected sound is relatively strong, the amplitude is high, and when the reflected sound is relatively weak, the amplitude is low, and thus this method is advantageous for distance measurement, but is currently rarely used because an image changes even if the direction of a probe is slightly different.

M-mode is a modified form of A-mode and is a mode in which the distance to a moving reflector is displayed as a temporal change. This is used to designate a region of interest (ROI) in a 2D image as an M line and display changes in that area over time, and is mainly used to observe heart valves, and may also record fetal heart sounds, but is recently being replaced by the Doppler method.

B-mode is a method of displaying reflected sounds as the brightness of dots, and is currently used in most ultrasound imaging equipment. The brightness of each dot is proportional to the amplitude of a reflected signal. Recently, B-mode has been providing brightness levels of 256 or more, and is also a mode that displays organ movements in real time. A mode called 2D mode, which stands for B (brightness) mode, displays the cross-section of an object in real time on a screen in black and white shades, and is the most commonly used mode.

In addition, Doppler mode is a mode in which typically blood flow is measured by detecting the flow of red blood cells in blood vessels. Here, the principle that the wavelength becomes shorter when the red blood cells approach the probe and longer when they move away is used. Depending on the method of displaying the blood flow, there are color Doppler, pulse wave Doppler (PW), and continuous wave Doppler (CW), etc. Doppler images may include blood flow Doppler images (also called color Doppler images) that show blood flow, tissue Doppler images that show tissue movement, and spectral Doppler images that show the speed of movement of an object as a waveform.

In addition, there is a composite mode in which two or three modes are applied to one image at the same time, displaying other modes together with 2D while mainly displaying 2D, and a 3D mode displaying a three-dimensional stereoscopic image.

In a process of processing B mode, B mode components are extracted from ultrasound data and processed, and in an image generation process, an ultrasound image in which the intensity of a signal is expressed as brightness may be generated based on the B mode components extracted during processing of B mode. In a Doppler processing process, Doppler components are extracted from ultrasound data, and in an image generation process, a Doppler image that expresses the movement of the object 10 in color or waveform may be generated based on the extracted Doppler components.

In the image generation process, a two-dimensional ultrasound image or a three-dimensional image of the object may be generated, and an elastic image that visualizes the degree of deformation of the object 10 according to pressure may also be generated. Furthermore, various additional information may be expressed in text and graphic format on the ultrasound image. Meanwhile, the generated ultrasound images may be stored in a memory.

In the process of measuring an object in an ultrasound image, a measuring tool for measuring the object may be determined, and one of a plurality of measuring tools may be selected based on a user input.

For example, a measurement tool selection menu may be provided for selecting one of a plurality of measurement tools, and the measurement tool selection menu may be displayed on a single screen together with an ultrasound image. Additionally, the measurement tool selection menu may be displayed on a separate screen from a touch screen on which the ultrasound image is displayed.

Additionally, one of the plurality of measurement tools may be determined based on a user input selecting one of the plurality of measurement items to be measured. The measurement items may include, but are not limited to, length, width, or angle.

Upon receiving a user input for selecting one of the measurement items, a predetermined measurement tool may be determined to correspond to the selected measurement item.

The image processor 130 may generate a time intensity curve representing image signal values for respective frames of an ultrasound image within a set region of interest. Specifically, the image processor 130 may extract image signal values for pixels of a region of interest in an ultrasound image, for example, brightness values of pixels in the region of interest, and digitize the values, and calculate the sum and average of the brightness values of respective pixels for respective ultrasound image frames. The image processor 130 may generate a graph showing the average of the brightness values of pixels within the calculated region of interest for a frame of an ultrasound image, and generate a time intensity curve (TIC) based on the graph. The time intensity curve is mainly used in ultrasound examinations using ultrasound contrast agents.

Errors may occur, such as a probe not coming into contact with an object, an object moving out of a region of interest, or the size of the region of interest changing, which will cause defects in the reading based on the time intensity curve. When it is determined that an error such as the above has occurred, the time intensity curve may be obtained again.

The image processor 130 may analyze the trend of the time intensity curve based on a predefined mathematical model and generate a graph for a frame of the ultrasound image based on a value according to the analyzed trend. In an embodiment, the predefined mathematical model may include at least one of a Polynomial, Exponential rise, Gamma variant and Gompertz model. The image processor 130 may generate a fitting curve representing the trend of a time intensity curve based on a user input selecting at least one of the Polynomial, Exponential rise, Gamma variant, and Gompertz models. However, the present disclosure is not limited thereto, and the image processor 130 may also generate a fitting curve based on a preset mathematical model among the mathematical models listed above. The technique of generating a fitting curve representing the tendency of a time intensity curve based on at least one of the Polynomial, Exponential rise, Gamma variant and Gompertz models is a technique known to those skilled in the art, and thus a detailed description thereof will be omitted.

The display 140 may display the generated ultrasound image and various information processed by the ultrasound imaging device 100. The ultrasound imaging device 100 may include one or more displays 140 depending on the implementation form. Additionally, the display 140 may be combined with a touch panel to be implemented as a touch screen.

The controller 120 may control the overall operation of the ultrasound imaging device 100 and a signal flow between internal components of the ultrasound imaging device 100. The controller 120 may include a memory that stores a program or data for performing a function of the ultrasound imaging device 100, and a processor that processes a program or data. Additionally, the controller 120 may receive a control signal from the input unit 170 or an external device to control the operation of the ultrasound imaging device 100.

The ultrasound imaging device 100 may include the communicator 160 and be connected to an external device (e.g., a server, a medical device, a portable device (smartphone, tablet PC, wearable device, etc.)) through the communicator 160.

The communicator 160 may include one or more components that enable communication with an external device, and may include, for example, at least one of a short-range communication module, a wired communication module, and a wireless communication module.

The communicator 160 may transmit and receive control signals and data from an external device, and transmit the received control signals to the controller 120 so that the controller 120 may control the ultrasound imaging device 100 according to the received control signals.

Alternatively, the controller 120 may control an external device according to a control signal of the controller by transmitting the control signal to the external device through the communicator 160.

For example, the external device may process data of the external device according to the control signal from the controller received through the communicator 160. An external device may be installed with a program (artificial intelligence, etc.) capable of controlling the ultrasound imaging device 100, and this program may include commands for performing part or all of the operations of the controller 120.

The program may be pre-installed on the external device, or a user of the external device may download and install the program from a server that provides an application. The server providing the application may include a storage medium on which the program is stored.

Additionally, the program may include a storage medium of the server or a storage medium of a client device in a system including the server and the client device. Alternatively, when there is a third device (such as a smartphone, tablet PC, wearable device, etc.) that communicates with the server or the client device, a program product may include a storage medium of the third device. Alternatively, the program may include a S/W program itself that is transmitted from the server to the client device or the third device, or from the third device to the client device.

In this case, one of the server, the client device, and the third device may execute a program to perform the method according to the disclosed embodiments. Alternatively, two or more of the server, the client device, and the third device may execute the program to implement the method according to the disclosed embodiments in a distributed manner.

For example, a server (e.g., a cloud server or an artificial intelligence server, etc.) may execute a program stored on the server to control a client device in communication with the server to perform the method according to the disclosed embodiments.

The method of operating an ultrasound imaging device, according to an embodiment, may be implemented in the form of program commands that may be executed through various computer means and recorded on a computer-readable medium. The computer-readable medium may include program commands, data files, data structures, etc., alone or in combination. The program commands recorded on the medium may be those specifically designed and configured for the present disclosure or may be known and available to those skilled in the art of computer software. Examples of computer-readable recording media include magnetic media such as hard disks, floppy disks, and magnetic tape, optical media such as CD-ROMs and DVDs, magneto-optical media such as floptical disks, and hardware devices specifically configured to store and execute program instructions, such as ROM, RAM, and flash memory. Examples of program instructions include machine language code, such as that produced by a compiler, as well as high-level language code that may be executed by a computer using an interpreter, etc.

Additionally, the ultrasound imaging device and the operating method of the ultrasound imaging device according to the disclosed embodiments may be provided as included in a computer program product. Computer program products may be traded between sellers and buyers as commodities.

A computer program product may include a S/W program and a computer-readable storage medium having the S/W program stored thereon. For example, the computer program product may include a product in the form of a S/W program (e.g., a downloadable app) distributed electronically by a manufacturer of an electronic device or through an electronic marketplace (e.g., Google Play Store, App Store). For electronic distribution, at least a portion of the S/W program may be stored on a storage medium or temporarily created. In this case, the storage medium may be a storage medium of a manufacturer's server, an electronic market server, or a relay server that temporarily stores SW programs.

The storage 150 may store various data or programs for driving and controlling the ultrasound imaging device 100, input/output ultrasound data, obtained ultrasound images, etc.

The input unit 170 may receive a user input for controlling the ultrasound imaging device 100. For example, a user input may include, but is not limited to, an input of manipulating buttons, keypads, mouses, trackballs, jog switches, knobs, etc., an input of touching a touchpad or touchscreen, a voice input, a motion input, a biometric input (e.g., iris recognition, fingerprint recognition, etc.), etc.

The block diagram of the ultrasound imaging device 100 illustrated in FIG. 1 is a block diagram for an embodiment, and each component of the block diagram of FIG. 1 may be integrated, added, or omitted depending on the specifications of the ultrasound imaging device 100 actually implemented. That is, two or more components may be combined into one component, or one component may be divided into two or more components, as needed. In addition, the functions performed in respective blocks are intended to explain embodiments, and the specific operations or devices thereof do not limit the scope of the present disclosure.

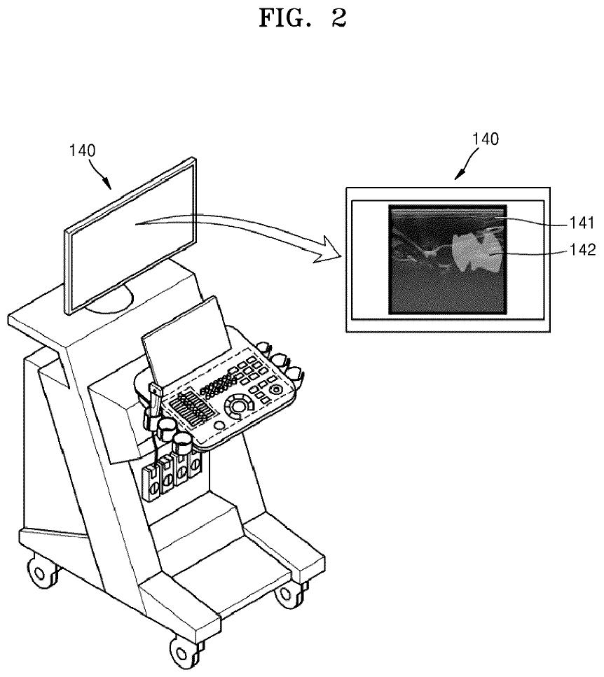

FIG. 2 is an enlarged view of a display including an area of a display area, in which an ultrasound image generated based on an ultrasound signal generated from a transducer of an ultrasound probe is output, according to an embodiment of the present disclosure.

Referring to FIG. 2, the display 140 displays an ultrasound image 141. Here, the display 140 may display, with color or a line, an area 142 with which a foreign substance has come into contact on the ultrasound image.

The display 140 is formed as a touch screen, and may display a user interface screen and receive a user input, for example, a touch, through the displayed user interface screen. The display 140 may be implemented in various manners, such as electrostatic capacitance, pressure, infrared, or ultrasound.

The display 140 may use a capacitive touch input method and may receive at least one touch through the user's body (e.g., a finger including the thumb) or a touch-capable input unit (e.g., a stylus pen, an electronic pen). An input applied to the display 140 may occur not only when the user physically contacts the display 140 with an input tool such as a finger or a stylus pen, but also when electrical contact is made by approaching the display 140 at a close distance without making contact (floating touch).

Meanwhile, a conductive foreign substance such as water, for example, may come into contact with (attach to) the display 140. In particular, when the ultrasound imaging device 100 is used, foreign substances such as ultrasound gel and disinfectant may frequently be on the display 140. Foreign substances such as the ultrasound gel may prevent a touch input from the display 140 or cause the display 140 to malfunction. Thus, the display 140 needs to display the location and size of the foreign substance so that the user may recognize the location of the foreign substance.

The controller 120 may check the electrostatic capacitance of the area of a region in which a touch is input on a panel of the display 140 or a region in which a foreign substance is located. As the storage 150 stores information about changes in electrostatic capacitance according to the contact of a foreign substance or input of a touch, the controller 120 may determine the input of a touch or contact of a foreign substance according to the change in electrostatic capacitance and display the location and size of the foreign substance on the display 140 by using a color or a line etc.

Meanwhile, the controller 120 may adjust the touch sensitivity of the display 140 for the area 142 with which a foreign substance has come into contact on the ultrasound image. For example, the controller 120 may determine that a foreign substance containing moisture such as water or sweat has come into contact with the display 140, and increase the touch sensitivity of the area 142 with which a foreign substance has come into contact so that a touch may be input even in the area 142 with which a foreign substance has come into contact.

In detail, the controller 120 may receive touch information (e.g., touch coordinates, touch time, or touch intensity, etc.) from the display 140. Additionally, the controller 120 may determine whether a touch input corresponding to the received touch information is a valid touch input. For example, the controller 120 may compare the touch intensity (e.g., the magnitude of a change in electrostatic capacitance) with a recognition criterion for a valid touch to determine whether a touch input is a valid touch input.

The controller 120 may include a function for determining the validity of a touch input differently by varying the touch sensitivity of the display 140 for the area 142 with which a foreign substance has come into contact on the ultrasound image. For example, when it is determined that a foreign substance has come into contact with the display 140, the controller 120 may increase the touch sensitivity of the display 140 to ensure that the user's touch is effectively input in the presence of foreign substances such as water or sweat. That is, the controller 120 may lower the magnitude of a change in electrostatic capacitance, processed as a valid touch input, and thus, even if a touch (weak touch) having a smaller magnitude than the change in electrostatic capacitance, processed as a valid touch and input before lowering (previously) is input in the area 142 with which a foreign substance has come into contact on the ultrasound image, the touch may be processed as a valid touch.

The controller 120 may adjust the touch sensitivity of the entire display 140, and may adjust the touch sensitivity only in the entire area in which the ultrasound image is displayed or in the area 142 with which a foreign substance on the ultrasound image has come into contact.

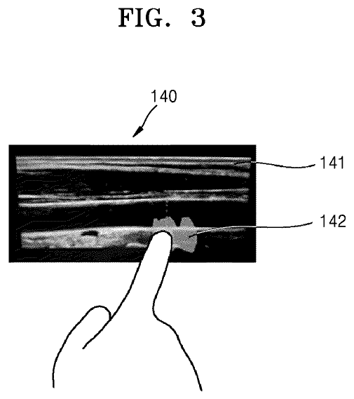

FIG. 3 is a diagram illustrating measurement on a display of an area in which an ultrasound image is output based on an ultrasound signal is output, according to an embodiment of the present disclosure.

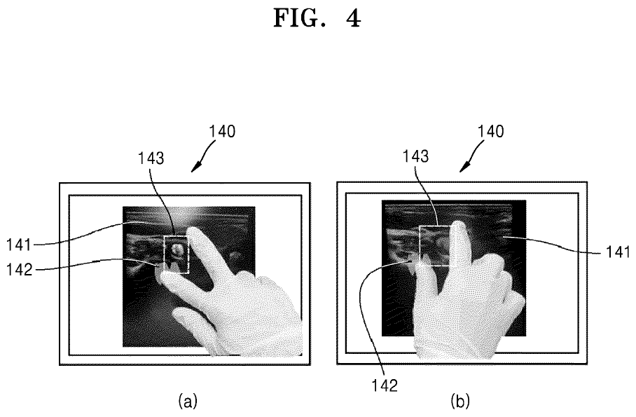

FIG. 4 is a diagram illustrating changing, on a display, of a region of interest of an area in which an ultrasound image generated based on an ultrasound signal is output, according to an embodiment of the present disclosure.

Referring to FIG. 3, the display 140 displays the ultrasound image 141 and the area 142 with which a foreign substance has come in contact on the ultrasound image, and measurement may be performed on the display 140.

Specifically, a touch may be performed to measure an object in the ultrasound image 141. Measurement items may include, but are not limited to, the length, width, or angle of a certain part of an organ or a certain bone. For example, two points on the display 140 may be touched and the length of a line connecting the two points may be measured, and points beyond that may be touched and the area or angle, etc. may be measured.

To facilitate measurement, an image of a measuring tool may be displayed on the ultrasound image 141. For example, an image of a length measuring tool for measuring the length of a certain part of an organ or a certain bone may be displayed. A user may intuitively recognize the measurement method through the image of the measuring tool. A position of the length measuring tool may be changed for convenience of measurement.

Referring to FIG. 4, the ultrasound imaging device 100 may set a region of interest 143 on the ultrasound image 141 based on positions of a plurality of measurement points and obtain measurement values for the set region of interest 143. The region of interest 143 may be resized or moved by touching and dragging the plurality of measurement points. Measurements may be performed within the region of interest 143.

For example, the ultrasound imaging device 100 may set a gate on the ultrasound image 141 based on the positions of two measurement points and measure a blood flow velocity in an area pointed to by the gate.

When it is determined that a foreign substance has come into contact with the display 140, the controller 120 may increase the touch sensitivity of the display 140 to allow the user's touch to be input effectively in the presence of foreign substances such as water or sweat. Accordingly, the size or position of the region of interest may be adjusted and measurement may be performed in the area 142 with which a foreign substance has come into contact on the ultrasound image.

The controller 120 may adjust the touch sensitivity immediately when a foreign substance comes into contact with the display 140, and the controller 120 may adjust the touch sensitivity of the display 140 only when a foreign substance comes into contact with the display 140 and further until a touch is input and the touch ends. However, the present disclosure is not limited thereto.

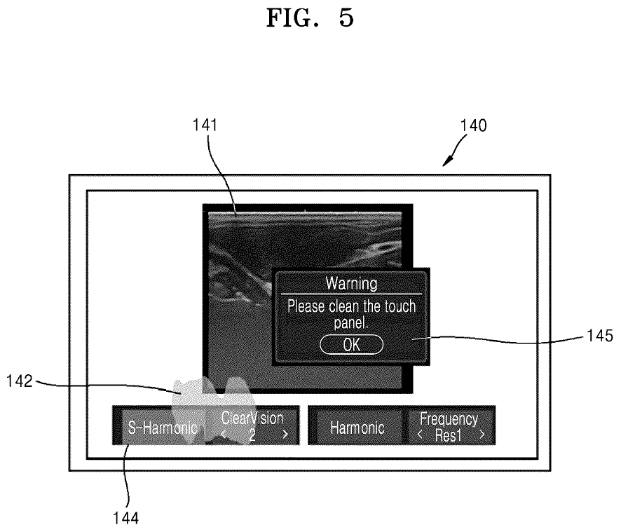

FIG. 5 is a diagram illustrating occurrence of a warning alarm in an ultrasound imaging device according to an embodiment of the present disclosure.

Referring to FIG. 5, the display 140 may further display one or more setting buttons 144 that may affect the ultrasound image. A plurality of setting buttons 144 may be included in a GUI of the display 140 and may include buttons that may adjust an ultrasound image. In FIG. 5, the setting buttons 144 are displayed below the ultrasound image 141, but the present disclosure is not limited thereto.

Considering the functions and operations provided by the ultrasound imaging device 100, the setting buttons 144 may be provided in a wide variety of types. For example, the setting buttons 144 may include buttons for adjusting the ultrasound image 141, such as Harmonic, TGC (Time Gain Compensation), Dual live (simultaneously displaying a 2D image and a Doppler image), Panoramic (panoramic view), MultiVision (using multi-beams to improve image quality), ClearVision (removing noise from an image), Freeze (pause), Save, B Mode (2D mode), C Mode (color mode), PW Mode (pulse wave Doppler mode—checking blood flow velocity), M Mode (Motion mode—drawing a virtual line in 2D and indicating movement corresponding to the line), Scan Area (adjusting the width of an image), Angle (adjusting the angle of an image), and Dynamic Range (adjusting the contrast by adjusting the ratio of the minimum and maximum values of an input signal). Additionally, the setting buttons 144 may include buttons for adjusting an interface, such as L/R Flip (switching a display mode of the GUI) and Top-Bottom Dual (switching the layout of input buttons of the display 140 from left-right to top-bottom).

For example, touching the Harmonic button may turn on/off the OHI (Optimal Harmonic Imaging) function, which optimizes an image using high frequencies, and touching the TGC button may adjust the gain according to the depth of an ultrasound image.

When a foreign substance comes into contact with the setting buttons 144 for adjusting the ultrasound image 141, the ultrasound image 141 may be adjusted unintentionally by the user. Thus, when it is determined that a foreign substance has come into contact with the setting buttons 144, the controller 120 may control the display 140 to pop up a warning alarm window 145. Here, the ultrasound image 141 may be automatically frozen.

As shown in FIG. 5, the warning alarm window 145 may include a phrase urging the user to clean the setting buttons 144 (e.g., Please clean the touch panel).

In addition, when it is determined that a foreign substance has come into contact with the setting buttons 144, the controller 120 may lower the touch sensitivity of the display 140 to prevent the user's touch from being input as a valid touch the by foreign substances such as water or sweat. That is, the controller 120 may increase the magnitude of a change in electrostatic capacitance, processed as a valid touch input, and thus even if a touch (strong touch) with a magnitude greater than the magnitude of the electrostatic capacitance change processed as a valid touch input before the increasing (previously) is input in the area 142 with which a foreign substance has come into contact on the ultrasound image, the controller 120 may process the touch as an invalid touch.

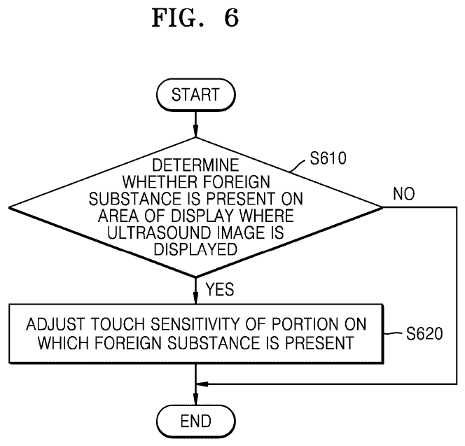

FIG. 6 is a flowchart of an operating method of an ultrasound imaging device, according to a first embodiment of the present disclosure.

Referring to FIG. 6, in operation S610, the controller 120 may determine whether the area of a region in which a touch is input on the panel of the display 140 is an area on an ultrasound image, and when at least a portion of the area on the ultrasound image includes a region in which a foreign substance is located, the controller 120 may check the electrostatic capacitance of at least a portion of the area on the ultrasound image.

As the storage 150 stores information about changes in electrostatic capacitance according to an input of touch or contact with a foreign substance, the controller 120 may determine the input of touch or contact with a foreign substance according to the change in electrostatic capacitance, and display, by using a color or line, etc., the location and size of the foreign substance on the ultrasound image of the display 140. When it is determined that no contact with a foreign substance has occurred, the controller 120 may not adjust the touch sensitivity on the ultrasound image of the display 140.

In operation S620, the controller 120 may adjust the touch sensitivity of the display 140 for the area 142 with which a foreign substance has come into contact on the ultrasound image. For example, the controller 120 may determine that a foreign substance containing moisture such as water or sweat has come into contact with the display 140, and increase the touch sensitivity of the area 142 with which the foreign substance has come into contact on the ultrasound image, so that a touch may be input even in the area 142 with which a foreign substance has come into contact on the ultrasound image.

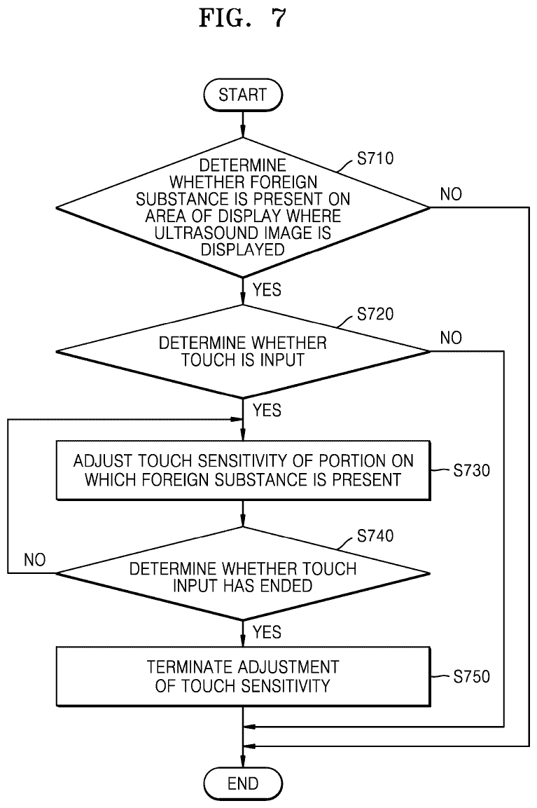

FIG. 7 is a flowchart of an operating method of an ultrasound imaging device, according to a second embodiment of the present disclosure.

In operation S710, the controller 120 may determine whether there is contact with a foreign substance on the ultrasound image, based on a change in electrostatic capacitance. The location and size of foreign substance may be displayed on the display 140 by using a color or a line, etc. When it is determined that no foreign substance has come into contact on the ultrasound image, the controller 120 may not adjust the touch sensitivity of the display 140.

In operation S720, the controller 120 may determine whether a touch is input, based on a change in electrostatic capacitance. When it is determined that no touch input has been made, the controller 120 may not adjust the touch sensitivity of the display 140.

In operation S730, the controller 120 may adjust the touch sensitivity in the area 142 with which a foreign substance has come into contact on the ultrasound image. The touch sensitivity may be increased for the area 142 with which a foreign substance has come into contact on the ultrasound image, and thus a touch may be input even in the area 142 with which a foreign substance has come into contact on the ultrasound image.

In operation S740, the controller 120 may determine whether the touch input has ended. The controller 120 may adjust the touch sensitivity in the area 142 with which a foreign substance has come into contact on the ultrasound image, until the touch input ends.

In operation S750, the controller 120 may terminate the adjustment of touch sensitivity when the touch input is ended. That is, when it is determined that a foreign substance has come into contact with the display 140, the controller 120 may adjust the touch sensitivity while a touch is input.

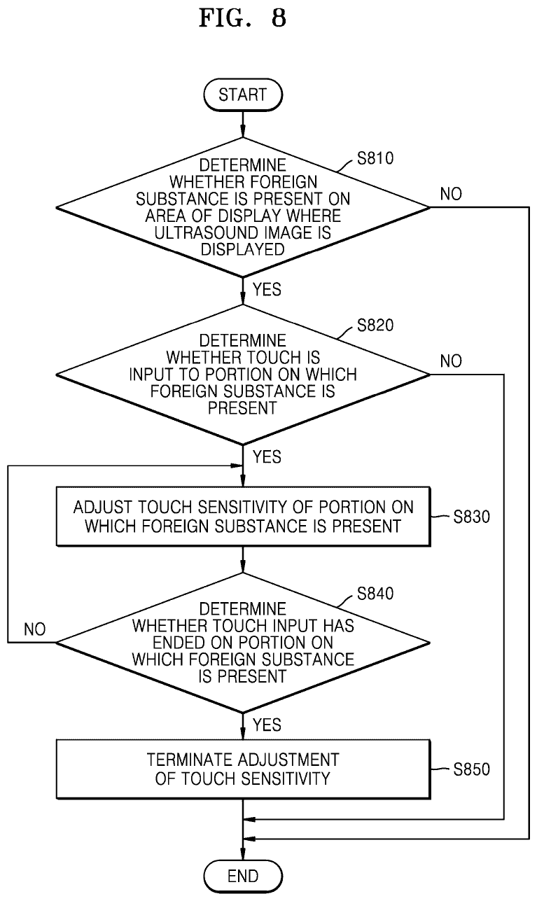

FIG. 8 is a flowchart of an operating method of an ultrasound imaging device, according to a third embodiment of the present disclosure. The details described with reference to FIG. 7 are omitted below.

In operation S820, the controller 120 may determine whether a touch is input, based on a change in electrostatic capacitance. When it is determined that no touch input has occurred in the area 142 with which a foreign substance has come into contact on the ultrasound image, the controller 120 may not adjust the touch sensitivity of the display 140.

In operation S830, the controller 120 may adjust the touch sensitivity in the area 142 with which a foreign substance has come into contact on the ultrasound image. The touch sensitivity may be increased for the area 142 with which a foreign substance has come into contact, so that touch may be input even in the area 142 with which a foreign substance has come into contact.

In operation S840, the controller 120 may determine whether the touch input has ended. The controller 120 may adjust the touch sensitivity in the area 142 with which a foreign substance has come into contact on the ultrasound image until the input of the touch ends in the area 142 with which a foreign substance has come into contact with the ultrasound image.

In operation S850, the controller 120 may terminate the adjustment of touch sensitivity when the touch input ends. That is, when it is determined that a foreign substance has come into contact with the display 140, the controller 120 may adjust the touch sensitivity while a touch is input to the area 142 with which a foreign substance has come into contact on the ultrasound image.

With reference to FIGS. 6 to 8, the controller 120 adjusting the touch sensitivity for the area in which the foreign substance is present on the ultrasound image (the area in which the foreign substance is in contact 142) is described. However, the touch sensitivity may be adjusted for the entire display 140 including the area in which the foreign substance is present on the ultrasound image or the entire area in which the ultrasound image is displayed.

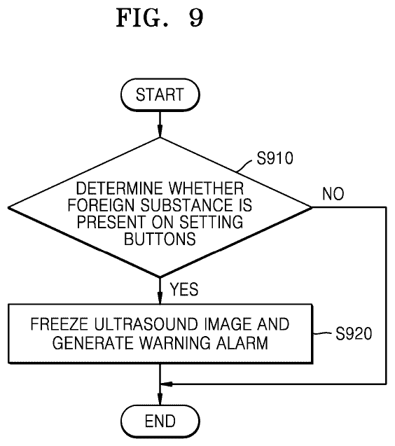

FIG. 9 is a flowchart of an operating method of an ultrasound imaging device, according to a fourth embodiment of the present disclosure.

In operation S910, the controller 120 may determine whether there is contact with a foreign substance, based on a change in electrostatic capacitance. The location and size of foreign substances may be displayed on the display 140 by using a color or a line, etc. When it is determined that no contact with a foreign substance has occurred, the controller 120 may not adjust the touch sensitivity of the display 140.

In operation S920, when it is determined that a foreign substance has come into contact with the setting buttons 144, the controller 120 may control the warning alarm window 145 to pop up on the display 140. Here, the ultrasound image 141 may be automatically frozen.

As described above, the disclosed embodiments have been described with reference to the attached drawings. It will be apparent to a person skilled in the art will that the present disclosure may be practiced in other forms than the disclosed embodiments without changing the technical spirit or essential characteristics of the present disclosure. The disclosed embodiments are illustrative and should not be construed as limiting.

1. An ultrasound imaging device comprising:
a display that displays an ultrasound image generated based on an ultrasound signal generated from a transducer of a probe and receives a user input through a touch screen; and
a controller that determines whether a foreign substance has come into contact with an area of the display where the ultrasound image is displayed,
wherein the controller adjusts touch sensitivity on the area of the display where the ultrasound image is displayed when it is determined that a foreign substance has come into contact with the area of the display.

2. The ultrasound imaging device of claim 1, wherein,
while a user input is received in an area with which the foreign substance has come into contact, the controller
adjusts the touch sensitivity on the area of the display where the ultrasound image is displayed, or
adjusts the touch sensitivity of an area that is in contact with the foreign substance.

3. The ultrasound imaging device of claim 1, wherein
the controller compares the user input received from the display with a recognition criterion for a valid touch to determine whether the user input is a valid input.

4. The ultrasound imaging device of claim 3, wherein
the recognition criterion for a valid input comprises a magnitude of a change in electrostatic capacitance.

5. The ultrasound imaging device of claim 4, wherein the controller
lowers the recognition criterion for a valid input and processes, as a valid input, a user input having a lower intensity than the recognition criterion for a valid input before the lowering on the ultrasound image.

6. The ultrasound imaging device of claim 3, wherein
the display further displays a setting button to adjust the ultrasound image,
wherein, when it is determined that a foreign substance has come into contact with the setting button, the controller freezes the ultrasound image or
causes a warning alarm window to pop up on the display.

7. The ultrasound imaging device of claim 6, wherein,
when it is determined that a foreign substance has come into contact with the setting button, the controller raises the recognition criterion for a valid input, and processes a user input on the setting button with an intensity greater than the recognition criterion for a valid input before the raising, as an invalid input.

8. An operating method of an ultrasound imaging device, the operating method comprising:
displaying an ultrasound image generated based on an ultrasound signal generated from a transducer of a probe and receiving a user input through a touch screen; and
determining whether a foreign substance has come into contact with an area of a display where the ultrasound image is displayed,
wherein, when it is determined that a foreign substance has come into contact with the area of the display where the ultrasound image is displayed, touch sensitivity on the area of the display is adjusted.

9. The operating method of claim 8, wherein,
while a user input is received in an area with which the foreign substance has come into contact,
the touch sensitivity on the area of the display where the ultrasound image is displayed is adjusted, or
the touch sensitivity of an area that is in contact with the foreign substance is adjusted.

10. The operating method of claim 8, wherein
the user input received from the display is compared with a recognition criterion for a valid touch to determine whether the user input is a valid input.

11. The operating method of claim 10, wherein
the recognition criterion for a valid input comprises a magnitude of a change in electrostatic capacitance.

12. The operating method of claim 11, wherein
the recognition criterion for a valid input is lowered and a user input having a lower intensity than the recognition criterion for a valid input before the lowering on the ultrasound image is processed as a valid input.

13. The operating method of claim 10, wherein
a setting button for adjusting the ultrasound image is further displayed, and
when it is determined that a foreign substance has come into contact with the setting button, the ultrasound image is frozen or
a warning alarm window is caused to pop up on the display.

14. The operating method of claim 13, wherein,
when it is determined that a foreign substance has come into contact with the setting button, the recognition criterion for a valid input is raised, and a user input on the setting button with an intensity greater than the recognition criterion for a valid input before the raising is processed as an invalid input.

15. A computer-readable recording medium including a program for executing an operating method of an ultrasound imaging device, wherein
the recording medium comprises the program for executing the operating method comprising:
displaying an ultrasound image generated based on an ultrasound signal generated from a transducer of a probe and receiving a user input through a touch screen; and
determining whether a foreign substance has come into contact with an area of the display where the ultrasound image is displayed,
wherein, when it is determined that a foreign substance has come into contact with the area of the display where the ultrasound image is displayed, touch sensitivity on the area of the display is adjusted.
